(19) United States
(12) Patent Application Publication
(10) Pub. No.: US 2023/0062272 A1
(43) Pub. Date: Mar. 2, 2023

(54) COMPOSITION FOR SIMULTANEOUSLY MODIFYING AMINO ACIDS OF SITE 736 AND SITE 738 OF PAPN GENE AND APPLICATION THEREOF

(71) Applicant: INSTITUTE OF ANIMAL SCIENCES OF CHINESE ACADEMY OF AGRICULTURAL SCIENCES, Beijing (CN)

(72) Inventors: Kui Li, Beijing (CN); Yulian Mu, Beijing (CN); Changjiang Xu, Beijing (CN); Zhiguo Liu, Beijing (CN)

(21) Appl. No.: 17/822,601

(22) Filed: Aug. 26, 2022

(30) Foreign Application Priority Data
Aug. 26, 2021 (CN) 202110985542.9

(51) Int. Cl. C12N 15/113; C12N 9/22; C12N 15/85

(52) U.S. Cl. CPC C12N 15/113; C12N 9/22; C12N 15/85; C12N 2310/20

(57) ABSTRACT

Provided are compositions for simultaneously modifying amino acids of site 736 and site 738 of pAPN gene and application thereof. An sgRNA set specifically recognizing porcine pAPN gene can recognize sequences near amino acids of site 736 and site 738 of pAPN gene, and has strong specificity. On this basis, in combination with a composition consisting of a cleavage protein and a double-stranded donor sequence, simultaneous modification of amino acids of site 736 and site 738 of the pAPN gene can be realized. Under editing of the composition, the amino acids of site 736 and site 738 of the pAPN gene are modified precisely and effectively, which can prevent normal expression of other amino acids of the pAPN gene from being damaged or changed. Therefore, it can resist TGEV infection, and also can retain the physiological activity function of the pAPN protein to the maximum extent.

### CROSS-REFERENCE TO RELATED APPLICATION

The present disclosure claims priority to Chinese patent application with the filing number 202110985542.9 filed on Aug. 26, 2021 with the Chinese Patent Office, and entitled “Composition for Simultaneously Modifying Amino Acids of Site 736 and Site 738 of pAPN Gene and Application thereof”, the contents of which are incorporated herein by reference in their entirety.

### SEQUENCE LISTING

The following application contains a sequence listing submitted electronically as a Standard ST.26 compliant XML file entitled “SequenceListing57742.xml,” created on Aug. 26, 2022, as 22,861 bytes in size, the entire contents of which are incorporated by reference herein.

### TECHNICAL FIELD

The present disclosure relates to the field of biotechnology, and in particular to a composition for simultaneously modifying amino acids of site 736 and site 738 of pAPN gene and application thereof.

### BACKGROUND ART

Porcine transmissible gastroenteritis (TGE) is an acute infectious intestinal disease, and has the characteristics of a high propagation speed and a high mortality rate. Once a pig is infected with this virus, the body weight and growth speed of the pig will be reduced. Clinically, the porcine transmissible gastroenteritis causes the most serious damage to suckling pigs.

The pAPN gene is one of cell surface receptors of the porcine transmissible gastroenteritis virus (TGEV), and has a CDS full length of 2892 bp, contains 21 exons, encodes 963 amino acids, and has a molecular weight about 150 kDa. See GenBank accession nos. HQ824547.1 or KU986724.1, incorporated by reference herein. GenBank accession HQ824547.1 coding sequence and corresponding protein is provided below for reference with mutation targets in bold.

| (SEQ ID NO: 11) | |
|---|---|
| 1 | atggccaagg gattctacat ttccaaggcc ctgggcatcc tgggcatcct cctcggcgtg |
| 61 | gcggccgtgg ccaccatcat cgctctgtct gtggtgtatg cccaggagaa gaacaagaat |
| 121 | gccgagcatg tcccccaagc ccccacgtcg cccaccatca ccaccacagc cgccatcacc |
| 181 | ttggaccaga gcaagccgtg gaaccggtac cgcctaccca caacgctgtt gcctgattcc |
| 241 | tacttcgtga cgctgagacc ctacctcact cccaacgcgg atggcctgta catcttcaag |
| 301 | ggcaaaagca tcgtccgctt actctgccag gagtccaccg atgtcatcat catccatagc |
| 361 | aagaagctca actacaccac ccaggggcac atggtggtcc tgcggggcgt gggggactcc |
| 421 | caggtcccag agatcgacag gactgagctg gtagagctca ctgagtacct ggtggtccac |
| 481 | ctcaagggct cgctgcagcc cggccacatg tacgagatgg agagtgaatt ccagggggaa |
| 541 | ctcgccgacg acctggcagg cttctaccgc agcgagtaca tggagggcaa cgtcaaaaag |
| 601 | gtgctggcca cgacacagat gcagtctaca gatgcccgga aatccttccc atgctttgac |
| 661 | gagccagcca tgaaggccac gttcaacatc actctcatcc accctaacaa cctcacggcc |
| 721 | ctgtccaata tgccgcccaa aggttccagc accccacttg cagaagaccc caactggtct |
| 781 | gacactgagt tcgaaaccac acctgtgatg tccacgtacc ttctggccta catcgtgagc |
| 841 | gagtcccaga gcgtgaatga aacggcccaa aatggcgtcc tgatccggat ctgggctcgg |
| 901 | cctaatgcaa ttgcagaggg ccatggcatg tatgccctga atgtgacagg tcccatccta |
| 961 | aacttctttg ccaatcatta taatacatcc tacccactcc ccaaatccga ccagattgcc |
| 1021 | ttgcccgact tcaatgccgg tgccatggag aactgggggc tggtgaccta ccgggagaac |
| 1081 | gcgctgctgt ttgacccaca gtcctcctcc atcagcaaca aagagcgagt tgtcactgtg |
| 1141 | attgctcacg aactggccca ccagtggttt ggcaacctgg tgaccctggc ctggtggaat |
| 1201 | gacctgtggc tgaatgaggg ctttgcctcc tatgtggagt acctgggtgc tgaccacgca |
| 1261 | gagcccacct ggaatctgaa agacctcatc gtgccaggcg acgtgtaccg agtgatggct |
| 1321 | gtggatgctc tggcttcctc ccacctgctg accacccctg ctgaggaggt caacacacct |
| 1381 | gcccagatca gcgagatgtt tgactccatc tcctacagca agggagcctc ggttatcagg |
| 1441 | atgctctcca acttcctgac tgaggacctg ttcaaggagg gcctggcgtc ctacttgcat |
| 1501 | gcctttgcct atcagaacac cacctacctg gacctgtggg agcacctgca gaaggctgtg |
| 1561 | gatgctcaga cgtccatcag gctgccagac actgtgagag ccatcatgga tcgatggacc |
| 1621 | ctgcagatgg gcttccccgt catcaccgtg gacaccaaga caggaaacat ctcacagaag |
| 1681 | cacttcctcc tcgactccga atccaacgtc acccgctcct cagcgttcga ctacctctgg |
| 1741 | attgttccca tctcatctat taaaaatggt gtgatgcagg atcactactg gctgcgggat |
| 1801 | gtttcccaag cccagaatga tttgttcaaa accgcatcgg acgattgggt cttgctgaac |
| 1861 | gtcaacgtga caggctattt ccaggtgaac tacgacgagg acaactggag gatgattcag |
| 1921 | catcagctgc agacaaacct gtcggtcatc cctgtcatca atcgggctca ggtcatctac |
| 1981 | gacagcttca acctggccac tgcccacatg gtccctgtca ccctggctct ggacaacacc |
| 2041 | ctcttcctga acggagagaa agagtacatg ccctggcagg ccgccctgag cagcctgagc |
| 2101 | tacttcagcc tcatgttcga ccgctccgag gtctatggcc ccatgaagaa atacctcagg |
| 2161 | aagcaggtcg aacccctctt ccaacatttc gaaactctca ctaaaaactg gaccgagcgc |
| 2221 | ccagaaaatc tgatggacca gtacagtgag attaatgcca tcagcactgc ctgctccaat |
| 2281 | ggattgcctc aatgtgagaa tctggccaag acccttttcg accagtggat gagcgaccca |
| 2341 | gaaaataacc cgatccaccc caacctgcgg tccaccatct actgcaatgc catagcccag |
| 2401 | ggcggccagg accagtggga ctttgcctgg gggcagttac aacaagccca gctggtaaat |
| 2461 | gaggccgaca aactccgctc agcgctggcc tgcagcaacg aggtctggct cctgaacagg |
| 2521 | tacctggatt acaccctgaa cccggacctc attcggaagc aagacgccac ctccactatt |
| 2581 | aacagcattg ccagcaatgt catcgggcag cctctggcct gggattttgt ccagagcaac |
| 2641 | tggaagaagc tctttcagga ctatggcggt ggttccttct ccttctccaa cctcattcag |
| 2701 | ggtgtgaccc gaagattctc ctctgagttt gagctgcagc agctggagca gttcaagaag |
| 2761 | aacaacatgg atgtgggctt cggctccggc acccgggctc tggagcaagc cctggagaag |
| 2821 | accaaggcca acatcaagtg ggtgaaggag aacaaggagg tggtgttgaa ttggttcata |
| 2881 | gagcacagct aa |

(SEQ ID NO: 12)
MAKGFYISKALGILGILLGVAAVATIIALSVVYAQEKNKNAEHVPQAPTSPTITTTAAITLD
QSKPWNRYRLPTTLLPDSYFVTLRPYLTPNADGLYIFKGKSIVRLLCQESTDVIIIHSKKL
NYTTQGHMVVLRGVGDSQVPEIDRTELVELTEYLVVHLKGSLQPGHMYEMESEFQGE
LADDLAGFYRSEYMEGNVKKVLATTQMQSTDARKSFPCFDEPAMKATFNITLIHPNNL
TALSNMPPKGSSTPLAEDPNWSDTEFETTPVMSTYLLAYIVSESQSVNETAQNGVLIRI
WARPNAIAEGHGMYALNVTGPILNFFANHYNTSYPLPKSDQIALPDFNAGAMENWGLV
TYRENALLFDPQSSSISNKERVVTVIAHELAHQWFGNLVTLAWWNDLWLNEGFASYVE
YLGADHAEPTWNLKDLIVPGDVYRVMAVDALASSHLLTTPAEEVNTPAQISEMFDSISY
SKGASVIRMLSNFLTEDLFKEGLASYLHAFAYQNTTYLDLWEHLQKAVDAQTSIRLPDT
VRAIMDRWTLQMGFPVITVDTKTGNISQKHFLLDSESNVTRSSAFDYLWIVPISSIKNGV
MQDHYWLRDVSQAQNDLFKTASDDVLLNVNVTGYFQVNYDEDNWRMIQHQLQTN
LSVIPVINRAQVIYDSFNLATAHMVPVTLALDNTLFLNGEKEYMPWQAALSSLSYFSLM
FDRSEVYGPMKKYLRKQVEPLFQHFETLTKNWTERPENLMDQYSEINAISTACSNGLP
QCENLAKTLFDQWMSDPENNPIHPNLRSTIYCNAIAQGGQDQWDFAWGQLQQAQLV
NEADKLRSALACSNEVWLLNRYLDYTLNPDLIRKQDATSTINSIASNVIGQPLAWDFVQ
SNWKKLFQDYGGGSFSFSNLIQGVTRRFSSEFELQQLEQFKKNNMDVGFGSGTRALE
QALEKTKANIKVWKENKEVVLNWFIEHS

During TGEV infection, positions where specific binding of S-protein of the virus to the receptor pAPN occurs are amino acids of sites 717-813 on the pAPN. In current research, in order to reduce the TGEV infection, a method of inhibiting or blocking the expression of the pAPN gene by gene editing is mostly used, and this method can effectively prevent and control attack and transfer of the virus. However, the pAPN gene also participates in other physiological processes, for example, cell growth, signal transduction, immunomodulation, angiogenesis, etc. Although the current method hinders the TGEV infection, other physiological processes of the cells also may be affected at the same time.

In view of this, the present disclosure is specifically proposed.

### SUMMARY

An sgRNA set specifically recognizing a porcine pAPN gene, characterized by including pAPN-sgRNA-1 and pAPN-sgRNA-2;

a nucleotide sequence encoding the pAPN-sgRNA-1 is represented by SEQ ID NO:1; and

a nucleotide sequence encoding the pAPN-sgRNA-2 is represented by SEQ ID NO:2.

A composition for simultaneously modifying amino acids of site 736 and site 738 of pAPN gene, characterized by including a cleavage protein, a double-stranded donor sequence, and the preceding sgRNA set;

the sgRNA set guides the cleavage protein to target specific sites and perform cleavage;

codons encoding the amino acids of site 736 and site 738 of the pAPN gene are located between the sites recognized by the sgRNA set; and

the double-stranded donor sequence is used to replace N736 of the pAPN gene with A736, and T738 with V738 (See e.g., SEQ ID NO:13).

A method of preparing a gene-edited cell, characterized in that the preceding composition is transferred into a target cell, to obtain a gene-edited cell.

### BRIEF DESCRIPTION OF DRAWINGS

In order to more clearly illustrate embodiments of the present disclosure or technical solutions in the prior art, accompanying drawings which need to be used in the description of the embodiments or the prior art will be introduced briefly below. Apparently, the accompanying drawings in the description below are for some embodiments of the present disclosure. Those ordinarily skilled in the art still could obtain other accompanying drawings according to these accompanying drawings, without using creative efforts.

FIG. 1 is a precise mutation pattern diagram of N736 and T738 double amino acids of a porcine pAPN gene provided in Example 1 of the present disclosure;

FIG. 2 is a sequencing result diagram of a porcine ileal epithelial cell with precisely modified amino acids of site 736 and site 738 of the pAPN gene provided in Example 2 of the present disclosure;

FIG. 3 is a qRT-PCR detection result diagram for TGEV-infection resistance of the porcine ileal epithelial cell with precisely modified amino acids of site 736 and site 738 of the pAPN gene provided in Example 2 of the present disclosure;

FIG. 4 is an IFA detection result diagram for TGEV-infection resistance of the porcine ileal epithelial cell with precisely modified amino acids of site 736 and site 738 of the pAPN gene provided in Example 2 of the present disclosure;

FIG. 5 is a TCID₅₀ detection result diagram for TGEV-infection resistance of the porcine ileal epithelial cell with precisely modified amino acids of site 736 and site 738 of the pAPN gene provided in Example 2 of the present disclosure; and

FIG. 6 is a sequencing result diagram of a porcine fibroblast with precisely modified amino acids of site 736 and site 738 of the pAPN gene provided in Example 3 of the present disclosure.

### DETAILED DESCRIPTION OF EMBODIMENTS

Embodiments of the present disclosure will be described in detail below in combination with examples, while those skilled in the art could understand that the following examples are merely for illustrating the present disclosure, but should not be considered as limitation on the scope of the present disclosure. If no specific conditions are specified in the examples, they are carried out under normal conditions or conditions recommended by the manufacturer.

Unless otherwise stated, professional and scientific terms used herein have the same meanings as those familiar to those skilled in the art. In addition, any method or material similar or equivalent to the contents described also can be used in the present disclosure.

A first objective of the present disclosure is to provide an sgRNA set specifically recognizing a porcine pAPN gene.

A second objective of the present disclosure is to provide a composition for simultaneously modifying amino acids of site 736 and site 738 of a pAPN gene.

A third objective of the present disclosure is to provide application of an sgRNA set or a composition in pAPN gene editing.

A fourth objective of the present disclosure is to provide a gene-edited cell and a preparation method thereof.

A fifth objective of the present disclosure is to provide a method of preparing a gene-edited pig.

In order to realize the above objectives of the present disclosure, the following technical solutions are particularly adopted.

An sgRNA set specifically recognizing a porcine pAPN gene, including pAPN-sgRNA-1 and pAPN-sgRNA-2;

a nucleotide sequence encoding the pAPN-sgRNA-1 is represented by SEQ ID NO:1; and

a nucleotide sequence encoding the pAPN-sgRNA-2 is represented by SEQ ID NO:2.

A composition for simultaneously modifying amino acids of site 736 and site 738 of pAPN gene, including a cleavage protein, a double-stranded donor sequence, and the preceding sgRNA set;

the sgRNA set guides the cleavage protein to target specific sites and perform cleavage;

codons encoding the amino acids of site 736 and site 738 of the pAPN gene are located between the sites recognized by the sgRNA set; and

the double-stranded donor sequence is used to replace N736 of the pAPN gene with A736 and replace T738 with V738.

Further, a solution where N736 is replaced with A736 is to replace a codon AAC encoding N736 with GCT, and a solution where T738 is replaced with V738 is to replace a codon ACC encoding T738 with GTC;

preferably, the double-stranded donor sequence includes a nucleotide sequence represented by SEQ ID NO:3.

Further, the cleavage protein includes Cas9, Cas9n, Cpf1 or C2c2, preferably Cas9;

preferably, a target cleavage vector 1 expresses the cleavage protein and the pAPN-sgRNA-1, and a target cleavage vector 2 expresses the cleavage protein and the pAPN-sgRNA-2;

preferably, vector skeletons of the target cleavage vector 1 and the target cleavage vector 2 are both independently CRISPR plasmids;

preferably, the CRISPR plasmids include CRISPR/Cas9, CRISPR/Cas9n, CRISPR/Cpf1 or CRISPR/C2c2, preferably CRISPR/Cas9; and

preferably, the CRISPR/Cas9 plasmid includes pX330, pX260, pX334, pX335, pX458, pX459, pX461, pX462, pX551 or pX552, preferably pX458.

Application of the above sgRNA set or composition in pAPN gene editing.

A method of preparing a gene-edited cell, wherein the above composition is transferred into a target cell, to obtain a gene-edited cell.

Further, the target cell includes a porcine fibroblast, and preferably includes a porcine fetal fibroblast; and

preferably, the transfer is carried out by electroporation or by liposome transfection.

The gene-edited cell prepared by the above preparation method.

A method of preparing a gene-edited pig, wherein a gene-edited cell is transplanted into an enucleated oocyte to obtain a recombinant cloned embryo, the recombinant cloned embryo is transplanted into a mother body to undergo pregnancy, to obtain a gene-edited pig with amino acids of site 736 and site 738 of a pAPN gene being modified simultaneously;

alternatively, the composition is microinjected into a porcine zygote-stage embryo by a microinjection method to obtain a pAPN gene modified embryo, and the gene modified embryo is transplanted into a mother body to undergo pregnancy to obtain a gene-edited pig with the amino acids of site 736 and site 738 of the pAPN gene being modified simultaneously.

Further, after birth, an identification step is further included for the gene-edited pig; and preferably, the identification includes sequencing identification.

Compared with the prior art, beneficial effects of the present disclosure are as follows.

The sgRNA set specifically recognizing the porcine pAPN gene provided in the present disclosure, and can precisely recognize the coding sequences near amino acids of site 736 and site 738 of the pAPN gene, and has strong specificity. On this basis, in combination with the composition consisting of the cleavage protein and the double-stranded donor sequence, simultaneous modification of amino acids of site 736 and site 738 of the pAPN gene can be realized. Under the guidance of the sgRNA set, the cleavage protein respectively recognizes two effect target spots, and realizes the sequence cleavage at the two effect target spots. The double-stranded donor sequence is used to replace the cleaved fragments, wherein codons encoding amino acids of site 736 and site 738 of the pAPN gene are located between the sites recognized by the sgRNA set, and after the replacement, the N736 of the pAPN gene is replaced with A736, and the T738 is replaced with V738. Under the editing of the composition, the amino acids of site 736 and site 738 of the pAPN gene are simultaneously modified precisely and effectively, which can prevent normal expression of other amino acids of the pAPN gene from being damaged or changed. Therefore, on the basis of resisting TGEV infection, the physiological activity function of the pAPN protein is retained to the maximum extent, and the composition has the advantages of a wide application range, high gene editing efficiency and so on.

The preparation methods of gene-edited cell and gene-edited pig provided in the present disclosure have simple operations, low costs, and strong universality, and the prepared gene-edited cell and gene-edited pig have good TGEV resistance.

The pAPN protein is a key receptor of TGEV entering cells, but besides playing an important role in mediating TGEV invasion, pAPN also plays a role in hydrolyzing amide bonds in structures such as peptide and amide in small intestine so as to release different N-neutral amino acids. Meanwhile, the pAPN protein also participates in many important physiological processes in other tissues, for example, cell growth, immune regulation, and blood pressure regulation. Thus, directly knocking out pAPN may affect other physiological functions of the body. Research results of amino acid point mutation of the present disclosure show that asparagine (N) at site 736 and threonine (T) at site 738 of exon 16 in the pAPN peptide chain are two most important amino acid sites affecting its activity as TGEV receptor.

Based on the above contents, the present disclosure provides an sgRNA set specifically recognizing a porcine pAPN gene, including pAPN-sgRNA-1 and pAPN-sgRNA-2, wherein a nucleotide sequence encoding the pAPN-sgRNA-1 is represented by SEQ ID NO:1, and a nucleotide sequence encoding the pAPN-sgRNA-2 is represented by SEQ ID NO:2.

This sgRNA set has strong specificity and high recognition efficiency.

The sequence encoding the pAPN-sgRNA-1:
(SEQ ID NO: 1)
CTAGAAATACCTCAGGAAGC;
and
the sequence encoding the pAPN-sgRNA-2:
(SEQ ID NO: 2)
CGAGCGCCCAGAAAATCTGA.

A composition for simultaneously modifying amino acids of site 736 and site 738 of pAPN gene, including a cleavage protein, a double-stranded donor sequence, and the sgRNA set of the present disclosure; the sgRNA set guides the cleavage protein to target specific sites and perform cleavage; codons encoding the amino acids of site 736 and site 738 of the pAPN gene are located between the sites recognized by the sgRNA set; and the double-stranded donor sequence is used to replace N736 of the pAPN gene with A736, and T738 with V738.

Under the editing of the composition, the amino acids of site 736 and site 738 of the pAPN gene are modified precisely and effectively, and normal expression of other amino acids of the pAPN gene can be prevented from being damaged or changed. Therefore, on the basis of resisting TGEV infection, the physiological activity function of the pAPN protein is retained to the maximum extent, the composition has the advantages of a wide application range, high gene editing efficiency and so on, and a powerful support can be provided for preparing and breeding new anti-TGEV pig breeds with precise mutation of double amino acids of the pAPN.

It should be noted that, the cleavage protein is not specifically limited herein, as long as the function of cleaving the target sequence under the guidance of the sgRNAs can be realized, for example, the cleavage protein may be Cas9, Cas9n, Cpf1 or C2c2, etc.

In a preferred embodiment, the solution where N736 is replaced with A736 is to replace a codon AAC encoding N736 with GCT, and the solution where T738 is replaced with V738 is to replace a codon ACC encoding T738 with GTC, and further preferably, the double-stranded donor sequence includes a nucleotide sequence represented by SEQ ID NO:3.

The pAPN-dsODN sequence is represented as follows:

(SEQ ID NO: 3)
cctttgagcacagtctggccttgtgcgaggcctttagcctctggcctcttg
ctcctgtagccattagctcttgctacatctgcccacccacatcagaggctc
catgggtctccagatgactcaggcatgagtctcttctttgaagctattttt
agggctgcatcctcggcatgtggaggttcccaagctaggggttgaatcgga
gctgtagccgccagcctacaccacagccacagcaacacgggatccgagcca
catctgcgacctacaccacagctcacagcaatgccagatccttaacccact
gagtggggccagggttgaacccatgtcctcatgtttcccagtcagattcgt
ttctgctgtgccatgacgggaactctggaacttcctctttgaagctcttta
tgttttgttcttgttttttgtttttgtttttctagaaatacctcaggaagc
aAgtcgaacccctcttccaacatttcgaaactctcactaaaGCTtggGTCg
agcgcccagaaaaCTtAatggaccagtgagtatgagctcgcttggtctgga
gatcatgggtggtgcaggtagcctgacctgggggcccatagcaagtccagc
agcatcctctctggagctcccaactcctggccggaccagggccacagtcag
ggagagcgacccctcccaaccccactcccggccccaggagtagggactctg
ctctgaggctctgtgtggcctatgaaccatctggcctctttgggcaaagga
ccaaactgaacctctgagggtccctcacccgcatggtgaggttctaggtgt
taaagctggggctggagcctgtgccagccctccccaggctgcccaagggca
agaagcaaagaagggaacccaaaggtggctggtgggctatacctgcagagt
gcgggtctgcctccctgttgggagttgtgtgtcagcaggggagtcttggtc
agcgtcaggtccaggcgtgctgacagagtgt.

In a preferred embodiment, the cleavage protein includes Cas9, Cas9n, Cpf1 or C2c2, preferably Cas9; the target cleavage vector 1 expresses the cleavage protein and the pAPN-sgRNA-1, the target cleavage vector 2 expresses the cleavage protein and the pAPN-sgRNA-2, and vector skeletons of the target cleavage vector 1 and the target cleavage vector 2 both are CRISPR plasmids.

In a preferred embodiment, the CRISPR plasmids include CRISPR/Cas9, CRISPR/Cas9n, CRISPR/Cpf1 or CRISPR/C2c2, preferably CRISPR/Cas9; the CRISPR/Cas9 plasmid includes pX330, pX260, pX334, pX335, pX458, pX459, pX461, pX462, pX551 or pX552, preferably pX458.

CRISPR/Cas9 and pX458 have wide universality, relatively strong versatility, and relatively high product maturity, and using the CRISPR/Cas9 and pX458 as gene editing vector skeleton can achieve higher enzyme digestion efficiency.

In a preferred embodiment, oligonucleotide single strands represented by sequences SEQ ID NOS: 4-5 and SEQ ID NOS: 6-7 are respectively annealed to form double stands, which are linked to the enzymatically-cleaved vector skeleton, respectively, and positive clones are screened out to obtain the target cleavage vector 1 and the target cleavage vector 2.

The pAPN-sgRNA-1-F sequence is represented by:
(SEQ ID NO: 4)
caccgCTAGAAATACCTCAGGAAGC;
The pAPN-sgRNA-1-R sequence is represented by:
(SEQ ID NO: 5)
aaacGCTTCCTGAGGTATTTCTAGc;
the pAPN-sgRNA-2-F sequence is represented by:
(SEQ ID NO: 6)
caccgCGAGCGCCCAGAAAATCTGA;
and
the pAPN-sgRNA-2-R sequence is represented by:
(SEQ ID NO: 7)
aaacTCAGATTTTCTGGGCGCTCGc.

Application of the sgRNA set or the composition in pAPN gene editing. The sgRNA set can realize specific recognition of a target site, and the composition can realize precise modification of amino acids of site 736 and site 738 of the pAPN gene. After N736 and T738 are precisely replaced with A736 and V738, binding of pAPN to TGEV can be blocked.

The present disclosure provides a gene-edited cell and a preparation method thereof, wherein the composition of the present disclosure is transferred into a target cell, to obtain a gene-edited cell. Further, a gene-edited cell is obtained by screening and identification. In the above, the target cell may be a porcine fibroblast, and is further preferably a porcine fetal fibroblast (the porcine fetal fibroblast has higher cloning efficiency than other cells); and a transfer method may be electroporation or liposome transfection.

The present disclosure further provides a method of preparing a gene-edited pig, wherein the gene-edited cell of the present disclosure is transplanted into an enucleated oocyte to obtain a recombinant cloned embryo, or a pAPN gene modified embryo is obtained by a microinjection method, and the recombinant cloned embryo or the gene modified embryo is transplanted into a mother body to undergo pregnancy, to obtain a gene-edited pig with amino acids of site 736 and site 738 of the pAPN gene being modified simultaneously. Further, the gene-edited pig is subjected to sequencing identification.

An identification method in the present disclosure is preferably: extracting DNA of a sample (cell or pig), performing PCR amplification by using primers represented by SEQ ID NOS: 8-9, sequencing an amplified product, and making a judgment according to a sequencing result.

The pAPN-TY-F2 sequence is represented by:
(SEQ ID NO: 8)
CAAGGATTTGTGGAGGAGAA;
and
the pAPN-TY-R2 sequence is represented by:
(SEQ ID NO: 9)
GCTGAGCGGAGTTTGTCG.

The present disclosure is further illustrated below with specific examples, but it should be understood that these examples are merely for more detailed description and should not be construed as limiting the present disclosure in any form.

Main reagents and instrument information used in the examples of the present disclosure are as follows.

Main reagents comprise collagenase type IV for isolating porcine fetal fibroblasts, purchased from sigma; DMEM, FBS, PS, NEAA, Glutamine, and Trypsase for cell culture, all purchased from Gibco; DNA kit for extracting cells and ear tissues, purchased from Tiangen Biotec (Beijing) Co., Ltd.; primers, synthesized by Tsingke Biotechnology Co., Ltd.; and KOD FX PCR enzyme for PCR, purchased from TOYOBO.

Main instrument comprises CO₂ incubator (Thermo Scientific, 3111); fluorescence inverted microscope (ZEISS, observer A1); PCR instrument (BIO-RID, C1000 Touch); gel imaging system (BIO-RID, Universal Hood II); Micromanipulator System (Eppendorf, Celltram vario); flow cytometry sorter (BD, Aria III).

### Example 1 Construction of Recombinant Plasmid pX458-pAPN-sgRNA Vector and Design of Double-Stranded Donor Sequence

1. First, sequences near the coding sequence of amino acids 736 and 738 of the pig pAPN gene are locked, and a target site close to sites N736 and T738 and meanwhile having a relatively high score is selected using sgRNA analysis tool CRISPOR (crispor.tefor.net), sequences thereof are represented by SEQ ID NO:1 (CTAGAATACCTCAGGAAGC) and SEQ ID NO:2 (CGAGCGCCAGAAAATCTGA). Complementarily paired oligonucleotide sequences represented by SEQ ID NO:4 (pAPN-sgRNA-1-F sequence: caccgCTAGAAATACCTCAGGAAGC) and SEQ ID NO:5 (pAPN-sgRNA-1-R sequence: aaacGCTTCCTGAGGTATTTCTAGc); and SEQ ID NO:6 (pAPN-sgRNA-2-F sequence: caccgCGAGCGCCCAGAAAATCTGA) and SEQ ID NO:7 (CD sgRNA-2-R sequence: aaacTCAGATTTTCTGGGCGCTCGc) are synthesized according to targeting site sequences. Meanwhile, according to sgRNA sequence, the present example also designs a dsODN sequence, as the double-stranded donor sequence, represented by SEQ ID NO:3 (pAPN-dsODN sequence: cctttgagcacagtctggccttgtgcgaggcctttagcctctggcctcttgctcctgtagccattagctcttgctacatctgccca cccacatcagaggctccatgggtctccagatgactcaggcatgagtctcttctttgaagctatttttagggctgcatcctcggc atgtggaggttcccaagctaggggttgaatcggagctgtagccgccagcctacaccacagccacagcaacacgggatc cgagccacatctgcgacctacaccacagctcacagcaatgccagatccttaacccactgagtggggccagggttgaac ccatgtcctcatgtttcccagtcagattcgtttctgctgtgccatgacgggaactctggaacttcctctttgaagctctttatgttttg ttcttgttttttgtttttgtttttctagaaatacctcaggaagcaAgtcgaacccctcttccaacatttcgaaactctcactaaaGC TtggGTCgagcgcccagaaaaCTtAatggaccagtgagtatgagctcgcttggtctggagatcatgggtggtgcagg tagcctgacctgggggcccatagcaagtccagcagcatcctctctggagctcccaactcctggccggaccagggccaca gtcagggagagcgacccctcccaaccccactcccggccccaggagtagggactctgctctgaggctctgtgtggcctatg aaccatctggcctctttgggcaaaggaccaaactgaacctctgagggtccctcacccgcatggtgaggttctaggtgttaa agctggggctggagcctgtgccagccctccccaggctgcccaagggcaagaagcaaagaagggaacccaaaggtg gctggtgggctatacctgcagagtgcgggtctgcctccctgttgggagttgtgtgtcagcaggggagtcttggtcagcgtca ggtccaggcgtgctgacagagtgt), and after the double-stranded donor sequence replaces the wild-type sequence, N736 and T738 are successfully replaced with A736 and V738. A precise mutation pattern diagram of double amino acids of N736 and T738 of the porcine pAPN gene is as shown in FIG. 1.

2. The constructed CRISPR/Cas9 recombinant plasmids targeting sequences near the amino acids of site 736 and site 738 of the pAPN gene are named as pX458-pAPN-sgRNA-1 and pX458-pAPN-sgRNA-2. The synthesized oligonucleotide was treated at 98° C. for 10 min, and then naturally cooled to room temperature and annealed; a pX458 skeleton vector containing Cas9 sequence was subjected to restriction enzyme digestion with restriction endonuclease Bbs I for 2 h under the condition of 37° C., a linearized fragment was recovered by gel cutting and then ligated with annealed oligonucleotide at 16° C. for 1 h, subsequently transformed Top10 or DH5α competent cell, the resultant was spread on an ampicillin-containing LB flat plate to grow, and a single colony was picked, cultured, and sequenced. A sequencing primer is U6-FWD (U6-FWD: GAGGGCCTATTTCCCATGATT) represented by SEQ ID NO:10. If the sequence was correct, after the expanding culture, the pX458-pAPN-sgRNA-1 plasmid and the pX458-pAPN-sgRNA-2 plasmid were extracted using the method provided on an Endo-Free Plasmid Maxi Kit, and the plasmids extracted were used for cell transfection.

### Example 2 Establishment and Functional Validation of Porcine Ileal Epithelial Cell Monoclones with Precisely Modified Amino Acids of Site 736 and Site 738 of pAPN Gene

1. Establishment of Porcine Ileal Epithelial Cells with Precisely Modified Amino Acids of Site 736 and Site 738 of pAPN Gene

Wild-type porcine ileal epithelial cells (Immortal Pig Intestinal-2I wild type, IPI-2I-WT) were recovered to 10 cm plate two days in advance, and cell transfection could be performed when the cells reached 70-80% confluence. 5 μg of pX458-pAPN-sgRNA-1 plasmid, 5 μg of pX458-pAPN-sgRNA-2 plasmid, and 5 μg of pAPN-dsODN were co-transfected into IPI-2I-WT cells. The transfection step was performed strictly according to instructions of Basic Primary Nucleofector Kit (Lonza).

After 36 h of electrotransformation, the cells were collected, individual positive cells were sorted by flow cytometry sorter into 96-well plate and cultured, and the culture solution was changed every 3 days. The sorted cells were cultured for about 10 days, and the cells in the 96-well plate could be observed to overgrow, then the monoclonal cells which overgrew were subcultured to a 48-well plate, and after the cells on the 48-well plate overgrew, a part of the cells were taken for extracting genome to identify the genotype.

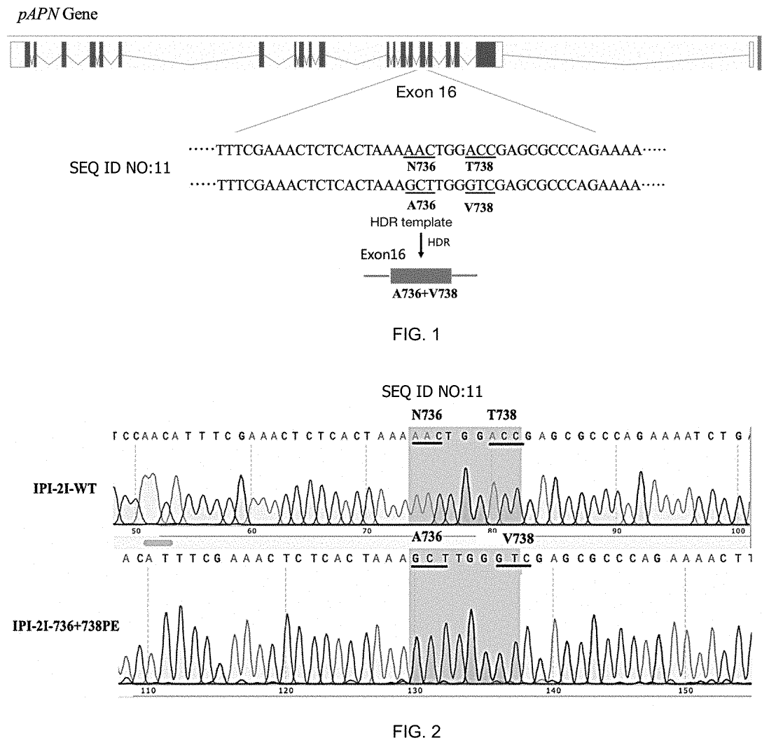

The picked cell monoclones were identified: taking the extracted cell genome as a template, amplifying the extracted DNA genome with upstream and downstream primers represented by nucleotide sequences such as SEQ ID NO:8 (pAPN-TY-F2 sequence: CAAGGATTTGTGGAGGAGAA) and SEQ ID NO:9 (pAPN-TY-R2 sequence: GCTGAGCGGAGTTTGTCG), to obtain a 1443 bp fragment through the amplification. The amplification condition was 94° C. for 5 min; 98° C. for 30 s, 62.6° C. for 30 s, 68° C. for 100 s, 34 cycles; 72° C. for 5 min. 2% agarose gel electrophoresis was performed to observe bands, and the PCR product was sent to Tsingke Biotechnology Co., Ltd. for sequencing. A cell sequencing result is as shown in FIG. 2, and IPI-2I (IPI-2I-736+738PE) cells with precisely modified amino acids of site 736 and site 738 of the pAPN gene were screened and used as donor cells in the TGEV infection test.

2. Functional Validation of Porcine Ileal Epithelial Cells with Precisely Modified Amino Acids of Site 736 and Site 738 of pAPN Gene

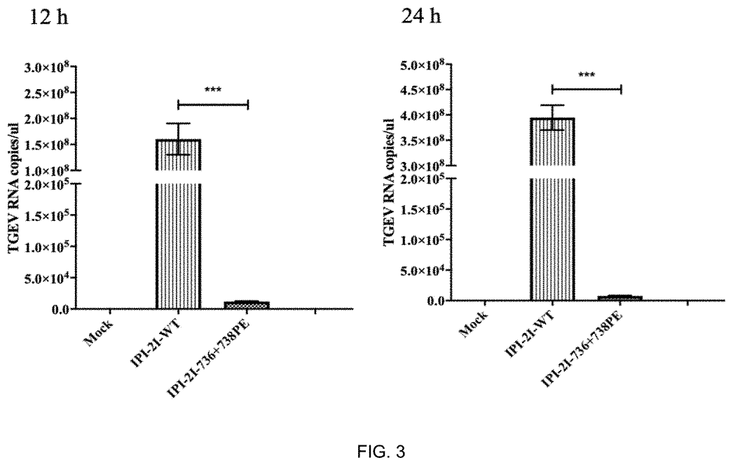

The IPI-2I-736+738PE cells obtained in the above were subjected to TGEV infection test. The number of copies of the TGEV genome in the cells was detected by using qRT-PCR. The TGEV virus strains (M01=1) were inoculated into IPI-2I-WT and IPI 736+738PE cells respectively. Mock group was a blank control group in which the IPI-2I-WT cells were not inoculated with the virus. After 12 h and 24 h of infection, cells were collected, and RNA was extracted to detect the number of TGEV virus copies in the cells. The qRT-PCR results are as shown in FIG. 3. The results indicate that the TGEV genome RNA replicates massively on the IPI-2I-WT cells, and compared with the IPI-2I-WT cells, the number of TGEV genome RNA copies in the IPI-2I-736+738PE cells is extremely significantly reduced (***P<0.001).

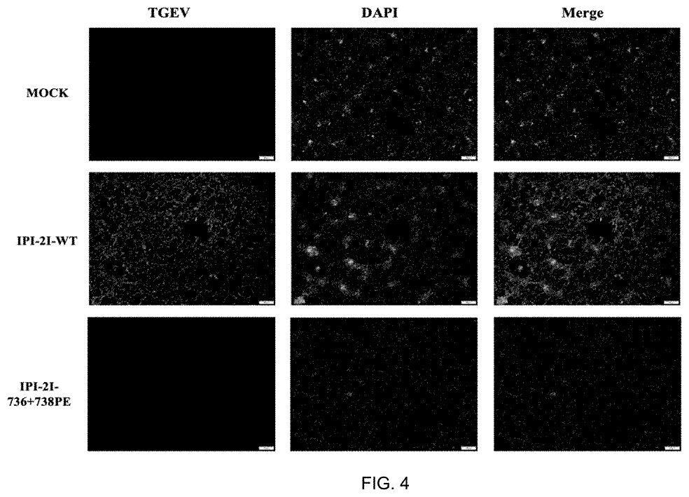

The TGEV infection situation in the cells was detected using indirect immunofluoresence (IFA), TGEV virus strains were inoculated into the IPI-2I-WT and IPI-2I-736+738PE cells (M01=1). Mock group was a blank control group in which the IPI-2I-WT cells were not inoculated with the virus. After 12 h of infection, indirect immunofluorescence assay was performed. The IFA results are as shown in FIG. 4. The results show that after the IPI-2I-WT cells were inoculated with the virus, TGEV in the cells were infected massively, and compared with the IPI-2I-WT cells, the IPI-2I-736+738PE cells had almost no TGEV infection.

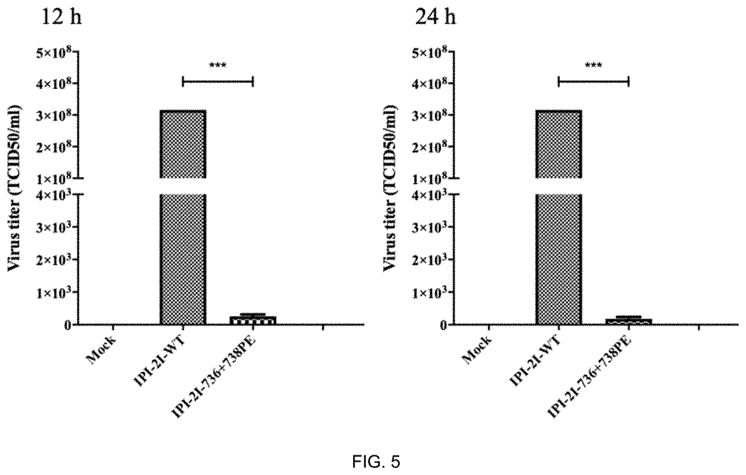

The TGEV virus titer in the cells was detected using TCID₅₀, TGEV virus strains were inoculated into the IPI-2I-WT and IPI-2I-736+738PE cells (M01=1). Mock group was a blank control group in which the IPI-2I-WT cells were not inoculated with the virus. After 12 h and 24 h of infection, a cell supernatant was collected, and subsequently the TGEV virus titer in the supernatant was tested using LLC-PK1 cells respectively. The TCID₅₀ results are as shown in FIG. 5. The results indicate that after the cells are inoculated with the virus, the TGEV virus titer in IPI-2I-WT cells is higher, and compared with IPI-2I-WT cells, the virus titer in the IPI-2I-736+738PE cells is extremely significantly reduced (***P<0.001).

The above results show that the porcine ileal epithelial cell with precisely modified amino acids of site 736 and site 738 of pAPN gene can effectively resist TGEV infection, indicating that the amino acids of site 736 and site 738 of the pAPN gene are key sites of TGEV infection, and the precisely modified amino acids of site 736 and site 738 of the pAPN gene can effectively resist TGEV infection.

### Example 3 Establishment of Porcine Fibroblasts with Precisely Modified Amino Acids of Site 736 and Site 738 of pAPN Gene

1. Preparation of Porcine Fetal Fibroblasts

The head, tail, limbs, internal organs, and bones of 35-day-old pig embryo were removed, and the blood was cleaned up. The fetus was continuously sheared for 30 min by using elbow ophthalmic scissors to ensure sufficient shearing, the sheared fetus tissues were pipetted into a 15 mL centrifuge tube by using a blue gun head of the scissors, 5 mL complete medium was added, after natural settling for several minutes, an upper solution was removed, several drops of fetal calf serum was added into underlying tissue block, the resultant was sucked out by using a 15 cm glass Pasteur tube bent at 1 cm of tip, and plated in two T75 culture flasks. The flasks were placed bottom up, and 15 mL complete medium was added at an opposite side. The culture flasks were carefully turned over after 6-8 h, to soak the tissue block into the culture solution. The solution was changed once every two days. After the cells overgrew in the T75 culture flasks, the cells were frozen and stored for later use. In the above, the pig is a pig raised in a pig farm of base of Institute of Animal Sciences of CAAS.

2. Cell Transfection

The primary porcine fetal fibroblasts were recovered to 10 cm plate one day before transfection, and cell transfection could be performed when the cells reached 70-80% confluence. 5 μg of pX458-pAPN-sgRNA-1 plasmid, 5 μg of pX458-pAPN-sgRNA-2 plasmid, and 5 μg of pAPN-dsODN were co-transfected into the porcine fetal fibroblasts. The transfection step was performed strictly according to instructions of Basic Primary Fibroblasts Nucleofector Kit (Lonza).

3. Screening of Positive Monoclonal Cells

After 36 h of electrotransformation, the cells were collected, individual positive cells were sorted by flow cytometry sorter into 96-well plate and cultured, and the culture solution was changed every 3 days. The sorted cells were cultured for about 10 days, and the cells in the 96-well plate could be observed to overgrow, then the monoclonal cells which overgrew were subcultured to a 48-well plate, and after the cells on the 48-well plate overgrew, a part of the cells were taken for extracting genome to identify the genotype.

4. Verification of Positive Monoclonal Cells

The picked cell monoclones were identified: taking the extracted cell genome as a template, amplifying the extracted DNA genome with upstream and downstream primers represented by nucleotide sequences SEQ ID NO:8 (pAPN-TY-F2 sequence: CAAGGATTTGTGGAGGAGAA) and SEQ ID NO:9 (pAPN-TY-R2 sequence: GCTGAGCGGAGTTTGTCG), to obtain a 1443 bp fragment through the amplification. The amplification condition was 94° C. for 5 min; 98° C. for 30 s, 62.6° C. for 30 s, 68° C. for 100 s, 34 cycles; 72° C. for 5 min. 2% agarose gel electrophoresis was performed to observe bands, and the PCR product was sent to Tsingke Biotechnology Co., Ltd. for sequencing. According to the sequencing, the porcine fibroblasts with precisely modified amino acids of site 736 and site 738 of the pAPN gene were screened as donor cells during nuclear transplantation.

5. Experiment Results

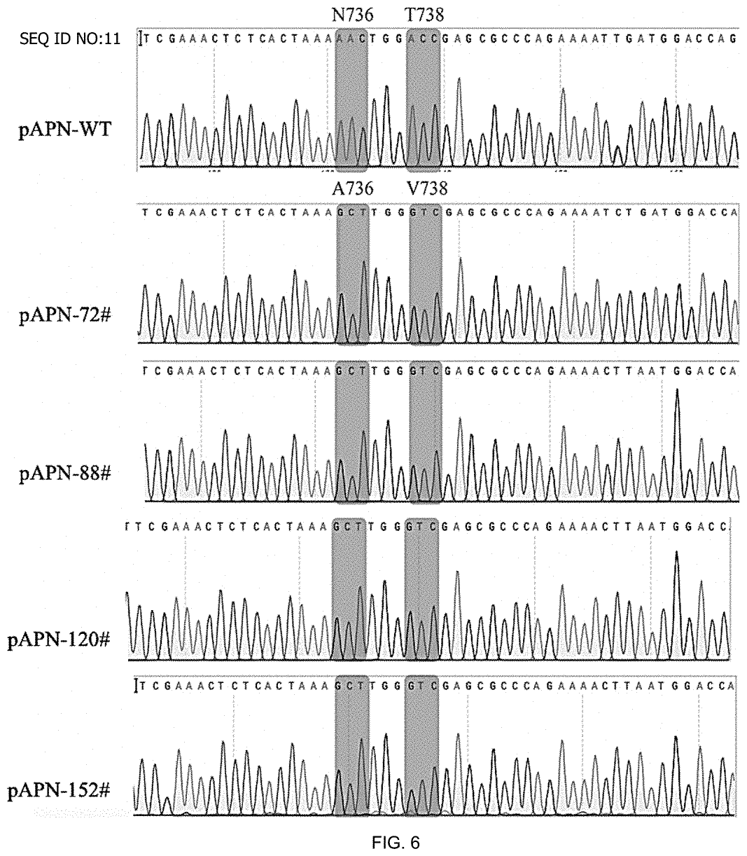

Sequencing results show that a plurality of porcine fibroblasts with precisely modified amino acids of site 736 and site 738 of the pAPN gene are successfully obtained in the present example, with the efficiency of 2.5%. The sequencing results of the positive cells are as shown in FIG. 6.

### Example 4 Preparation of Gene-Edited Pigs with Precisely Modified Amino Acids of Site 736 and Site 738 of pAPN Gene by Somatic Cell Nuclear Transplantation Technique

The positive cells edited by homozygous genes obtained in Example 3 were used as donor cells of nuclear transfer, enucleated porcine oocytes matured in vitro for 40 h was used as recipient cells of nuclear transfer, and the donor cells of nuclear transfer were transferred into the oocytes. Upon electrofusion and activation, recombinant cloned embryos were constructed. A cloned recombinant embryo with good development state was picked, and transferred into uterus of a naturally estrous multiparity Yorkshire (large white) sow by a surgical method to undergo pregnancy, wherein steps of embryo transfer of the surgical method are as follows: intravenously injecting Zoletil anesthetic into the recipient sow at an injection dosage of 5 mg/kg body weight for induced anesthesia, after anesthesia, moving the recipient sow to a surgery rack for supine fixation, and performing respiratory anesthesia (the concentration of isoflurane was 3%-4%), making a surgical incision about 10 cm long at ventrimeson of the recipient sow, to expose the ovary, the fallopian tube, and the uterus, making an embryo transplantation glass tube enter about 5 cm along the fimbria of fallopian tube, to transplant the cloned recombinant embryo in a good development state into a junction of the ampulla and the isthmus of the fallopian tube. After embryo transfer, the technician periodically observed and examined the recipient sow for pregnancy with type B ultrasound.

After birth of piglets, ear tissues were sheared and genomic DNA was extracted, to undergo PCR amplification using the above nucleotide sequences of SEQ ID NO:8 and SEQ ID NO:9. A PCR amplification product was sequenced to detect the genotype.

Although the present disclosure has been illustrated and described with specific examples, it should be aware that many other alterations and modifications can be made without departing from the spirit and scope of the present disclosure. Therefore, it means that the attached claims cover all these changes and modifications within the scope of the present disclosure.

### INDUSTRIAL APPLICABILITY

The sgRNA set specifically recognizing the porcine pAPN gene provided in the present disclosure can specifically recognize sequences near amino acids of site 736 and site 738 of the pAPN gene, and has strong specificity. On this basis, in combination with the composition consisting of the cleavage protein and the double-stranded donor sequence, simultaneous modification of amino acids of site 736 and site 738 of the pAPN gene can be realized. Under the guidance of the sgRNA set, the cleavage protein respectively recognizes two effect target spots, and realizes the sequence cleavage at the two effect target spots. The double-stranded donor sequence is used to replace the cleaved fragments, wherein codons encoding amino acids of site 736 and site 738 of the pAPN gene are located between the sites recognized by the sgRNA set, and after the replacement, the N736 of the pAPN gene is replaced with A736, and the T738 is replaced with V738. Under the editing of the composition, the amino acids of site 736 and site 738 of the pAPN gene are simultaneously modified precisely and effectively, which can prevent normal expression of other amino acids of the pAPN gene from being damaged or changed. Therefore, on the basis of resisting TGEV infection, the physiological activity function of the pAPN protein is retained to the maximum extent, and the composition has the advantages of a wide application range, high gene editing efficiency and so on.

The preparation methods of gene-edited cell and gene-edited pig provided in the present disclosure have simple operations, low costs, and strong universality, and the prepared gene-edited cell and gene-edited pig have good TGEV resistance.

What is claimed is:

1. An sgRNA set specifically recognizing a porcine pAPN gene, comprising pAPN-sgRNA-1 and pAPN-sgRNA-2, wherein
a nucleotide sequence encoding the pAPN-sgRNA-1 is represented by SEQ ID NO:1; and
a nucleotide sequence encoding the pAPN-sgRNA-2 is represented by SEQ ID NO:2.

2. A composition for simultaneously modifying amino acids of site 736 and site 738 of pAPN gene, comprising a cleavage protein, a double-stranded donor sequence, and the sgRNA set according to claim 1, wherein
the sgRNA set guides the cleavage protein to target specific sites and perform cleavage;
codons encoding the amino acids of site 736 and site 738 of the pAPN gene are located between the sites recognized by the sgRNA set; and
the double-stranded donor sequence is used to replace N736 of the pAPN gene with A736, and T738 with V738.

3. The composition according to claim 2, wherein a solution where N736 is replaced with A736 is to replace a codon AAC encoding N736 with GCT, and a solution where T738 is replaced with V738 is to replace a codon ACC encoding T738 with GTC.

4. The composition according to claim 2, wherein the cleavage protein comprises Cas9, Cas9n, Cpf1 and C2c2.

5. The composition according to claim 3, wherein the double-stranded donor sequence comprises a nucleotide sequence represented by SEQ ID NO: 3.

6. The composition according to claim 4, wherein the cleavage protein is Cas9.

7. The composition according to claim 4, wherein a target cleavage vector 1 expresses the cleavage protein and the pAPN-sgRNA-1, and a target cleavage vector 2 expresses the cleavage protein and the pAPN-sgRNA-2.

8. The composition according to claim 7, wherein vector skeletons of the target cleavage vector 1 and the target cleavage vector 2 are both independently a CRISPR plasmid.

9. The composition according to claim 8, wherein the CRISPR plasmid comprises CRISPR/Cas9, CRISPR/Cas9n, CRISPR/Cpf1 and CRISPR/C2c2.

10. The composition according to claim 9, wherein the CRISPR plasmid is CRISPR/Cas9 plasmid.

11. The composition according to claim 10, wherein the CRISPR/Cas9 plasmid comprises pX330, pX260, pX334, pX335, pX458, pX459, pX461, pX462, pX551 and pX552.

12. The composition according to claim 11, wherein the CRISPR/Cas9 plasmid is pX458.

13. The composition according to claim 7, wherein oligonucleotide single strands represented by sequences SEQ ID NOS: 4-5 and SEQ ID NOS: 6-7 are respectively annealed to form double stands, which are linked to enzymatically-cleaved vector skeleton, respectively, and positive clones are screened out to obtain the target cleavage vector 1 and the target cleavage vector 2.

14. A method of preparing a gene-edited cell, wherein the composition according to claim 2 is transferred into a target cell, to obtain a gene-edited cell.

15. The method according to claim 14, wherein the target cell comprises a porcine fibroblast.

16. The preparation method according to claim 15, wherein the target cell comprises a porcine fetal fibroblast.

17. The preparation method according to claim 15, wherein transfer is carried out by electroporation or by liposome transfection.


SEQUENCE LISTING (13 sequences)

SEQ ID NO: 1 - DNA, 20 bp, synthetic construct
  misc_feature 1..20: note = pAPN-sgRNA-1
  source 1..20: mol_type = other DNA
ctagaaatacctcaggaagc

SEQ ID NO: 2 - DNA, 20 bp, synthetic construct
  misc_feature 1..20: note = pAPN-sgRNA-2
  source 1..20: mol_type = other DNA
cgagcgcccagaaaatctga

SEQ ID NO: 3 - DNA, 1000 bp, synthetic construct
  misc_feature 1..1000: note = pAPN-dsODN sequence
  source 1..1000: mol_type = other DNA
cctttgagcacagtctggccttgtgcgaggcctttagcctctggcctcttgctcctgtagccattagctcttgctacatctgcccacccacatcagaggctccatgggtctccagatgactcaggcatgagtctcttctttgaagctatttttagggctgcatcctcggcatgtggaggttcccaagctaggggttgaatcggagctgtagccgccagcctacaccacagccacagcaacacgggatccgagccacatctgcgacctacaccacagctcacagcaatgccagatccttaacccactgagtggggccagggttgaacccatgtcctcatgtttcccagtcagattcgtttctgctgtgccatgacgggaactctggaacttcctctttgaagctctttatgttttgttcttgttttttgtttttgtttttctagaaatacctcaggaagcaagtcgaacccctcttccaacatttcgaaactctcactaaagcttgggtcgagcgcccagaaaacttaatggaccagtgagtatgagctcgcttggtctggagatcatgggtggtgcaggtagcctgacctgggggcccatagcaagtccagcagcatcctctctggagctcccaactcctggccggaccagggccacagtcagggagagcgacccctcccaaccccactcccggccccaggagtagggactctgctctgaggctctgtgtggcctatgaaccatctggcctctttgggcaaaggaccaaactgaacctctgagggtccctcacccgcatggtgaggttctaggtgttaaagctggggctggagcctgtgccagccctccccaggctgcccaagggcaagaagcaaagaagggaacccaaaggtggctggtgggctatacctgcagagtgcgggtctgcctccctgttgggagttgtgtgtcagcaggggagtcttggtcagcgtcaggtccaggcgtgctgacagagtgt

SEQ ID NO: 4 - DNA, 25 bp, synthetic construct
  misc_feature 1..25: note = pAPN-sgRNA-1-F
  source 1..25: mol_type = other DNA
caccgctagaaatacctcaggaagc

SEQ ID NO: 5 - DNA, 25 bp, synthetic construct
  misc_feature 1..25: note = pAPN-sgRNA-1-R
  source 1..25: mol_type = other DNA
aaacgcttcctgaggtatttctagc

SEQ ID NO: 6 - DNA, 25 bp, synthetic construct
  misc_feature 1..25: note = pAPN-sgRNA-2-F
  source 1..25: mol_type = other DNA
caccgcgagcgcccagaaaatctga

SEQ ID NO: 7 - DNA, 25 bp, synthetic construct
  misc_feature 1..25: note = pAPN-sgRNA-2-R
  source 1..25: mol_type = other DNA
aaactcagattttctgggcgctcgc

SEQ ID NO: 8 - DNA, 20 bp, synthetic construct
  misc_feature 1..20: note = pAPN-TY-F2
  source 1..20: mol_type = other DNA
caaggatttgtggaggagaa

SEQ ID NO: 9 - DNA, 18 bp, synthetic construct
  misc_feature 1..18: note = pAPN-TY-R2
  source 1..18: mol_type = other DNA
gctgagcggagtttgtcg

SEQ ID NO: 10 - DNA, 21 bp, synthetic construct
  misc_feature 1..21: note = U6-FWD
  source 1..21: mol_type = other DNA
gagggcctatttcccatgatt

SEQ ID NO: 11 - DNA, 2892 bp, Sus scrofa
  source 1..2892: mol_type = genomic DNA
  misc_feature 2206..2208: note = point mutation for N736A
  misc_feature 2212..2214: note = point mutation for T738V
atggccaagggattctacatttccaaggccctgggcatcctgggcatcctcctcggcgtggcggccgtggccaccatcatcgctctgtctgtggtgtatgcccaggagaagaacaagaatgccgagcatgtcccccaagcccccacgtcgcccaccatcaccaccacagccgccatcaccttggaccagagcaagccgtggaaccggtaccgcctacccacaacgctgttgcctgattcctacttcgtgacgctgagaccctacctcactcccaacgcggatggcctgtacatcttcaagggcaaaagcatcgtccgcttactctgccaggagtccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaactcgccgacgacctggcaggcttctaccgcagcgagtacatggagggcaacgtcaaaaaggtgctggccacgacacagatgcagtctacagatgcccggaaatccttcccatgctttgacgagccagccatgaaggccacgttcaacatcactctcatccaccctaacaacctcacggccctgtccaatatgccgcccaaaggttccagcaccccacttgcagaagaccccaactggtctgacactgagttcgaaaccacacctgtgatgtccacgtaccttctggcctacatcgtgagcgagtcccagagcgtgaatgaaacggcccaaaatggcgtcctgatccggatctgggctcggcctaatgcaattgcagagggccatggcatgtatgccctgaatgtgacaggtcccatcctaaacttctttgccaatcattataatacatcctacccactccccaaatccgaccagattgccttgcccgacttcaatgccggtgccatggagaactgggggctggtgacctaccgggagaacgcgctgctgtttgacccacagtcctcctccatcagcaacaaagagcgagttgtcactgtgattgctcacgaactggcccaccagtggtttggcaacctggtgaccctggcctggtggaatgacctgtggctgaatgagggctttgcctcctatgtggagtacctgggtgctgaccacgcagagcccacctggaatctgaaagacctcatcgtgccaggcgacgtgtaccgagtgatggctgtggatgctctggcttcctcccacctgctgaccacccctgctgaggaggtcaacacacctgcccagatcagcgagatgtttgactccatctcctacagcaagggagcctcggttatcaggatgctctccaacttcctgactgaggacctgttcaaggagggcctggcgtcctacttgcatgcctttgcctatcagaacaccacctacctggacctgtgggagcacctgcagaaggctgtggatgctcagacgtccatcaggctgccagacactgtgagagccatcatggatcgatggaccctgcagatgggcttccccgtcatcaccgtggacaccaagacaggaaacatctcacagaagcacttcctcctcgactccgaatccaacgtcacccgctcctcagcgttcgactacctctggattgttcccatctcatctattaaaaatggtgtgatgcaggatcactactggctgcgggatgtttcccaagcccagaatgatttgttcaaaaccgcatcggacgattgggtcttgctgaacgtcaacgtgacaggctatttccaggtgaactacgacgaggacaactggaggatgattcagcatcagctgcagacaaacctgtcggtcatccctgtcatcaatcgggctcaggtcatctacgacagcttcaacctggccactgcccacatggtccctgtcaccctggctctggacaacaccctcttcctgaacggagagaaagagtacatgccctggcaggccgccctgagcagcctgagctacttcagcctcatgttcgaccgctccgaggtctatggccccatgaagaaatacctcaggaagcaggtcgaacccctcttccaacatttcgaaactctcactaaaaactggaccgagcgcccagaaaatctgatggaccagtacagtgagattaatgccatcagcactgcctgctccaatggattgcctcaatgtgagaatctggccaagacccttttcgaccagtggatgagcgacccagaaaataacccgatccaccccaacctgcggtccaccatctactgcaatgccatagcccagggcggccaggaccagtgggactttgcctgggggcagttacaacaagcccagctggtaaatgaggccgacaaactccgctcagcgctggcctgcagcaacgaggtctggctcctgaacaggtacctggattacaccctgaacccggacctcattcggaagcaagacgccacctccactattaacagcattgccagcaatgtcatcgggcagcctctggcctgggattttgtccagagcaactggaagaagctctttcaggactatggcggtggttccttctccttctccaacctcattcagggtgtgacccgaagattctcctctgagtttgagctgcagcagctggagcagttcaagaagaacaacatggatgtgggcttcggctccggcacccgggctctggagcaagccctggagaagaccaaggccaacatcaagtgggtgaaggagaacaaggaggtggtgttgaattggttcatagagcacagctaa

SEQ ID NO: 12 - protein, 963 aa, Sus scrofa
MAKGFYISKALGILGILLGVAAVATIIALSVVYAQEKNKNAEHVPQAPTSPTITTTAAITLDQSKPWNRYRLPTTLLPDSYFVTLRPYLTPNADGLYIFKGKSIVRLLCQESTDVIIIHSKKLNYTTQGHMVVLRGVGDSQVPEIDRTELVELTEYLVVHLKGSLQPGHMYEMESEFQGELADDLAGFYRSEYMEGNVKKVLATTQMQSTDARKSFPCFDEPAMKATFNITLIHPNNLTALSNMPPKGSSTPLAEDPNWSDTEFETTPVMSTYLLAYIVSESQSVNETAQNGVLIRIWARPNAIAEGHGMYALNVTGPILNFFANHYNTSYPLPKSDQIALPDFNAGAMENWGLVTYRENALLFDPQSSSISNKERVVTVIAHELAHQWFGNLVTLAWWNDLWLNEGFASYVEYLGADHAEPTWNLKDLIVPGDVYRVMAVDALASSHLLTTPAEEVNTPAQISEMFDSISYSKGASVIRMLSNFLTEDLFKEGLASYLHAFAYQNTTYLDLWEHLQKAVDAQTSIRLPDTVRAIMDRWTLQMGFPVITVDTKTGNISQKHFLLDSESNVTRSSAFDYLWIVPISSIKNGVMQDHYWLRDVSQAQNDLFKTASDDWVLLNVNVTGYFQVNYDEDNWRMIQHQLQTNLSVIPVINRAQVIYDSFNLATAHMVPVTLALDNTLFLNGEKEYMPWQAALSSLSYFSLMFDRSEVYGPMKKYLRKQVEPLFQHFETLTKNWTERPENLMDQYSEINAISTACSNGLPQCENLAKTLFDQWMSDPENNPIHPNLRSTIYCNAIAQGGQDQWDFAWGQLQQAQLVNEADKLRSALACSNEVWLLNRYLDYTLNPDLIRKQDATSTINSIASNVIGQPLAWDFVQSNWKKLFQDYGGGSFSFSNLIQGVTRRFSSEFELQQLEQFKKNNMDVGFGSGTRALEQALEKTKANIKWVKENKEVVLNWFIEHS

SEQ ID NO: 13 - protein, 963 aa, Sus scrofa
  source 1..963: note = Mutated sequence
MAKGFYISKALGILGILLGVAAVATIIALSVVYAQEKNKNAEHVPQAPTSPTITTTAAITLDQSKPWNRYRLPTTLLPDSYFVTLRPYLTPNADGLYIFKGKSIVRLLCQESTDVIIIHSKKLNYTTQGHMVVLRGVGDSQVPEIDRTELVELTEYLVVHLKGSLQPGHMYEMESEFQGELADDLAGFYRSEYMEGNVKKVLATTQMQSTDARKSFPCFDEPAMKATFNITLIHPNNLTALSNMPPKGSSTPLAEDPNWSDTEFETTPVMSTYLLAYIVSESQSVNETAQNGVLIRIWARPNAIAEGHGMYALNVTGPILNFFANHYNTSYPLPKSDQIALPDFNAGAMENWGLVTYRENALLFDPQSSSISNKERVVTVIAHELAHQWFGNLVTLAWWNDLWLNEGFASYVEYLGADHAEPTWNLKDLIVPGDVYRVMAVDALASSHLLTTPAEEVNTPAQISEMFDSISYSKGASVIRMLSNFLTEDLFKEGLASYLHAFAYQNTTYLDLWEHLQKAVDAQTSIRLPDTVRAIMDRWTLQMGFPVITVDTKTGNISQKHFLLDSESNVTRSSAFDYLWIVPISSIKNGVMQDHYWLRDVSQAQNDLFKTASDDWVLLNVNVTGYFQVNYDEDNWRMIQHQLQTNLSVIPVINRAQVIYDSFNLATAHMVPVTLALDNTLFLNGEKEYMPWQAALSSLSYFSLMFDRSEVYGPMKKYLRKQVEPLFQHFETLTKAWVERPENLMDQYSEINAISTACSNGLPQCENLAKTLFDQWMSDPENNPIHPNLRSTIYCNAIAQGGQDQWDFAWGQLQQAQLVNEADKLRSALACSNEVWLLNRYLDYTLNPDLIRKQDATSTINSIASNVIGQPLAWDFVQSNWKKLFQDYGGGSFSFSNLIQGVTRRFSSEFELQQLEQFKKNNMDVGFGSGTRALEQALEKTKANIKWVKENKEVVLNWFIEHS